(19) United States
(12) Patent Application Publication
(10) Pub. No.: US 2025/0235312 A1
(43) Pub. Date: Jul. 24, 2025

(54) REPLACEMENT HEART VALVE SYSTEM, IMPLANT DELIVERY SYSTEM, AND METHOD OF DEPLOYING A REPLACEMENT HEART VALVE IMPLANT

(71) Applicant: BOSTON SCIENTIFIC SCIMED, INC., MAPLE GROVE, MN (US)

(72) Inventors: Donal Kyne, Galway (IE); Joseph Murphy, Galway (IE); Sean O'Sullivan, Galway (IE)

(73) Assignee: BOSTON SCIENTIFIC SCIMED, INC., MAPLE GROVE, MN (US)

(21) Appl. No.: 19/033,865

(22) Filed: Jan. 22, 2025

Related U.S. Application Data
(60) Provisional application No. 63/623,941, filed on Jan. 23, 2024.

(51) Int. Cl. A61F 2/24

(52) U.S. Cl. CPC A61F 2/2436

(57) ABSTRACT

An implant delivery system may include an elongate shaft assembly including an implant holding portion including a proximal sheath and a distal sheath. The shaft assembly may include an outer tubular member coupled to the proximal sheath, an intermediate tubular member, and an inner shaft coupled to the distal sheath. The intermediate tubular member may include a joint movably coupling proximal and distal portions of the intermediate tubular member. A replacement heart valve system may further include a replacement heart valve implant and a guidewire. A method of deploying the implant may include advancing the delivery system to a native heart valve, shifting the proximal sheath from a first position to a second position, deflecting the distal portion of the intermediate tubular member relative to the proximal portion at the joint, and thereafter, shifting the distal sheath from a first position to a second position.

### CROSS-REFERENCE TO RELATED APPLICATIONS

This application claims the benefit of priority of U.S. Provisional Application No. 63/623,941 filed Jan. 23, 2024, the entire disclosure of which is hereby incorporated by reference.

### TECHNICAL FIELD

The disclosure relates generally to medical devices and more particularly to medical devices that are adapted for implanting stents and medical devices including a stent component.

### BACKGROUND

A wide variety of intracorporeal medical devices have been developed for medical use including, artificial heart valves for repair or replacement of diseased heart valves. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In one example, an implant delivery system for delivering a replacement heart valve implant to a native heart valve may comprise an elongate shaft assembly including an implant holding portion comprising a proximal sheath and a distal sheath. The elongate shaft assembly may comprise an outer tubular member coupled to the proximal sheath, an intermediate tubular member slidably disposed within the outer tubular member, and an inner shaft coupled to the distal sheath and slidably disposed within the intermediate tubular member. The intermediate tubular member may comprise a joint disposed within the implant holding portion, the joint movably coupling a distal portion of the intermediate tubular member to a proximal portion of the intermediate tubular member.

In addition or alternatively to any example disclosed herein, the joint is configured to permit at least one degree of movement between the distal portion of the intermediate tubular member and the proximal portion of the intermediate tubular member when the distal portion of the intermediate tubular member is laterally unconstrained relative to the proximal portion of the intermediate tubular member.

In addition or alternatively to any example disclosed herein, the proximal sheath is configured to shift between a first position in which the proximal sheath laterally constrains the distal portion of the intermediate tubular member relative to the proximal portion of the intermediate tubular member, and a second position in which the proximal sheath does not laterally constrain the distal portion of the intermediate tubular member relative to the proximal portion of the intermediate tubular member.

In addition or alternatively to any example disclosed herein, the joint is configured to permit the distal portion of the intermediate tubular member to form an obtuse angle with the proximal portion of the intermediate tubular member when the distal portion of the intermediate tubular member is laterally unconstrained relative to the proximal portion of the intermediate tubular member.

In addition or alternatively to any example disclosed herein, the inner shaft comprises a lumen extending therethrough, the lumen being configured to slidably receive a guidewire therein.

In addition or alternatively to any example disclosed herein, the inner shaft extends through the joint.

In addition or alternatively to any example disclosed herein, the joint comprises a ball joint having a socket fixedly attached to the proximal portion of the intermediate tubular member and a ball fixedly attached to the distal portion of the intermediate tubular member.

In addition or alternatively to any example disclosed herein, the joint comprises a metallic tubular member extending between the proximal portion of the intermediate tubular member and the distal portion of the intermediate tubular member.

In addition or alternatively to any example disclosed herein, the metallic tubular member comprises a plurality of interconnected links cut from a monolithic tube.

In addition or alternatively to any example disclosed herein, a replacement heart valve system may comprise a replacement heart valve implant, a guidewire, and an implant delivery system for delivering the replacement heart valve implant to a native heart valve, the implant delivery system comprising an elongate shaft assembly including an implant holding portion comprising a proximal sheath and a distal sheath. In a closed configuration the implant holding portion may be configured to constrain the replacement heart valve implant in a radially collapsed configuration. The elongate shaft assembly may comprise an outer tubular member coupled to the proximal sheath, an intermediate tubular member slidably disposed within the outer tubular member, and an inner shaft coupled to the distal sheath and slidably disposed within the intermediate tubular member. The intermediate tubular member may comprise a joint disposed within the implant holding portion, the joint movably coupling a distal portion of the intermediate tubular member to a proximal portion of the intermediate tubular member. The guidewire may be slidably disposable within a lumen of the inner shaft.

In addition or alternatively to any example disclosed herein, the joint is configured to permit the distal portion of the intermediate tubular member to deflect laterally relative to the proximal portion of the intermediate tubular member when the distal portion of the intermediate tubular member is unconstrained by the proximal sheath.

In addition or alternatively to any example disclosed herein, the guidewire comprises a self-biased distal portion forming a spiral configuration when unconstrained.

In addition or alternatively to any example disclosed herein, when the self-biased distal portion of the guidewire is disposed within the lumen of the inner shaft and the distal portion of the intermediate tubular member is unconstrained by the proximal sheath, the distal portion of the intermediate tubular member is biased laterally by the self-biased distal portion of the guidewire.

In addition or alternatively to any example disclosed herein, the implant delivery system further comprises a proximal shuttle assembly disposed on the intermediate tubular member distal of the joint, the proximal shuttle assembly extending radially outward from the intermediate tubular member.

In addition or alternatively to any example disclosed herein, engagement of the proximal shuttle assembly with the proximal sheath substantially prevents deflection of the distal portion of the intermediate tubular member relative to the proximal portion of the intermediate tubular member at the joint.

In addition or alternatively to any example disclosed herein, a method of deploying a replacement heart valve implant within a native heart valve of a patient's heart may comprise advancing an implant delivery system to a position adjacent the native heart valve, the implant delivery system comprising an elongate shaft assembly including an implant holding portion comprising a proximal sheath and a distal sheath configured to retain the replacement heart valve implant in a radially collapsed configuration, wherein the elongate shaft assembly comprises an outer tubular member coupled to the proximal sheath, an intermediate tubular member slidably disposed within the outer tubular member, and an inner shaft coupled to the distal sheath and slidably disposed within the intermediate tubular member, and wherein the intermediate tubular member comprises a joint disposed within the implant holding portion, the joint movably coupling a distal portion of the intermediate tubular member to a proximal portion of the intermediate tubular member; shifting the proximal sheath from a first position to a second position, thereby releasing a proximal portion of the replacement heart valve implant; deflecting the distal portion of the intermediate tubular member relative to the proximal portion of the intermediate tubular member at the joint; and thereafter, shifting the distal sheath from a first position to a second position, thereby releasing a distal portion of the replacement heart valve implant.

In addition or alternatively to any example disclosed herein, the distal portion of the intermediate tubular member is substantially prevented from deflecting relative to the proximal portion of the intermediate tubular member when the proximal sheath is disposed in the first position.

In addition or alternatively to any example disclosed herein, the distal portion of the intermediate tubular member is substantially prevented from deflecting relative to the proximal portion of the intermediate tubular member at the joint when the proximal sheath is disposed in the first position.

In addition or alternatively to any example disclosed herein, the method may further comprise positioning a guidewire comprising a self-biased distal portion forming a spiral configuration when unconstrained such that the self-biased distal portion is disposed within a left ventricle of the patient's heart; and thereafter, advancing the implant delivery system over the guidewire to the position adjacent the native heart valve, wherein the guidewire is disposed within a lumen of the inner shaft.

In addition or alternatively to any example disclosed herein, the method may further comprise after shifting the proximal sheath from the first position to the second position, withdrawing at least a portion of the self-biased distal portion of the guidewire into a lumen of the inner shaft, wherein the self-biased distal portion of the guidewire is configured to deflect the distal portion of the intermediate tubular member laterally relative to the proximal portion of the intermediate tubular member at the joint.

In addition or alternatively to any example disclosed herein, rotation of the guidewire relative to the implant delivery system changes a direction of deflection of the distal portion of the intermediate tubular member relative to the proximal portion of the intermediate tubular member at the joint.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates selected aspects of a replacement heart valve implant;

FIG. 2 illustrates selected aspects of a replacement heart valve system;

FIG. 3 is a partial cross-sectional view illustrating selected aspects of an implant delivery system of the replacement heart valve system;

FIGS. 4-5 are partial cross-sectional views illustrating selected aspects of the implant delivery system of the replacement heart valve system;

FIGS. 6-7 are partial cross-sectional views illustrating selected aspects of the implant delivery system of the replacement heart valve system; and

FIGS. 8-12 schematically illustrate selected aspects of a method of deploying a replacement heart valve implant within a native heart valve.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

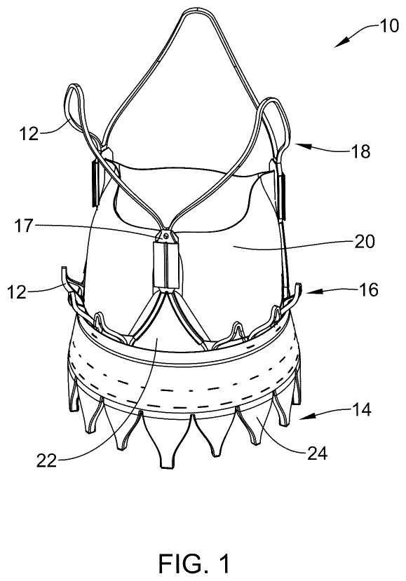

FIG. 1 illustrates selected aspects of a replacement heart valve implant 10. It should be appreciated that the replacement heart valve implant 10 can be any type of replacement heart valve (e.g., a mitral valve, an aortic valve, etc.). In use, the replacement heart valve implant 10 may be implanted (e.g., surgically or through transcatheter delivery) in a mammalian heart. The replacement heart valve implant 10 can be configured to allow one-way flow through the replacement heart valve implant 10 from an inflow end to an outflow end.

For the purpose of this disclosure, the discussion herein is directed toward use in treating a native heart valve such as the aortic valve and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

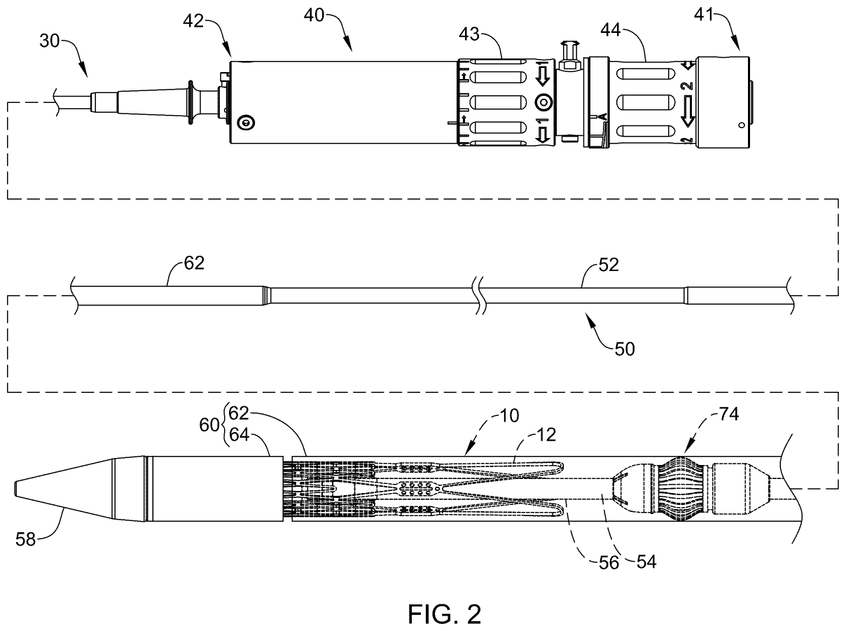

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The expandable framework 12 and/or the replacement heart valve implant 10 may be configured to shift between a radially collapsed configuration (e.g., FIG. 2) and a radially expanded configuration (e.g., FIG. 1). In some embodiments, the expandable framework 12 may be self-expanding from the radially collapsed configuration to the radially expanded configuration. In some embodiments, the expandable framework 12 may be self-biased toward the radially expanded configuration. In some alternative embodiments, the expandable framework 12 may be mechanically expandable from the radially collapsed configuration to the radially expanded configuration. In some alternative embodiments, the expandable framework 12 may be balloon expandable from the radially collapsed configuration to the radially expanded configuration. Other configurations are also contemplated. In some embodiments, the expandable framework 12 may include and/or define a plurality of interstices (e.g., openings) through the expandable framework 12.

In some embodiments, the expandable framework 12 may include and/or define a lower crown 14 proximate an inflow end, an upper crown 16 proximate an outflow end, and a plurality of stabilization arches 18 extending downstream from the outflow end. In some embodiments, the lower crown 14 may be disposed at the inflow end. In some embodiments, the upper crown 16 may be disposed at the outflow end. In some embodiments, the expandable framework 12 may include a tubular wall defining the central lumen, the inflow end, the outflow end, the lower crown 14, and/or the upper crown 16.

In some embodiments, the expandable framework 12 may include and/or define a plurality of commissure posts 17 proximate the outflow end. In some embodiments, the plurality of commissure posts 17 may at least partially define the outflow end. Other configurations are also contemplated. In some embodiments, the plurality of commissure posts 17 may be disposed longitudinally and/or axially between the upper crown 16 and the plurality of stabilization arches 18. In some embodiments, the plurality of stabilization arches 18 may extend downstream of and/or away from the upper crown 16 and/or the plurality of commissure posts 17 in a direction opposite the lower crown 14. In some embodiments, the upper crown 16 may be disposed longitudinally and/or axially between the lower crown 14 and the plurality of stabilization arches 18. In some embodiments, the upper crown 16 may be disposed longitudinally and/or axially between the lower crown 14 and the plurality of commissure posts 17.

In some embodiments, the replacement heart valve implant 10 may include a proximal portion and a distal portion. In some embodiments, orientation of the replacement heart valve implant 10 may be related to an implant delivery device and/or a direction of implantation relative to a treatment site (e.g., a native heart valve, the aortic valve, etc.). In some embodiments, the proximal portion may include the outflow end and/or the plurality of stabilization arches 18. In some embodiments, the proximal portion may include the plurality of commissure posts 17, the upper crown 16, and/or the plurality of valve leaflets 20. In some embodiments, the distal portion may include the inflow end and/or the lower crown 14. Other configurations are also contemplated.

In some embodiments, the replacement heart valve implant 10 may include a plurality of valve leaflets 20 disposed within the central lumen. The plurality of valve leaflets 20 may be coupled, secured, and/or fixedly attached to the expandable framework 12. In at least some embodiments, the plurality of valve leaflets 20 may be coupled, secured, and/or fixedly attached to the expandable framework 12 at the plurality of commissure posts 17 to form and/or define a plurality of commissures.

Each of the plurality of valve leaflets 20 may include a root edge coupled to the expandable framework 12 and a free edge (e.g., a coaptation edge) movable relative to the root edge to coapt with the free edges of the other valve leaflets along a coaptation region. In some embodiments, the plurality of valve leaflets 20 can be integrally formed with each other, such that the plurality of valve leaflets 20 is formed as a single unitary and/or monolithic unit. In some embodiments, the plurality of valve leaflets 20 may be formed integrally with other structures such as an inner skirt 22 and/or an outer skirt 24, base structures, liners, or the like.

The plurality of valve leaflets 20 may be configured to substantially restrict fluid from flowing through the replacement heart valve implant 10 in a closed position. For example, in some embodiments, the free edges of the plurality of valve leaflets 20 may move into coaptation with one another in the closed position to substantially restrict fluid from flowing through the replacement heart valve implant 10. The free edges of the plurality of valve leaflets 20 may be moved apart from each other in an open position to permit fluid flow through the replacement heart valve implant 10. In FIG. 1, the plurality of valve leaflets 20 is shown in the open position or in a partially open position (e.g., a neutral position) that the plurality of valve leaflets 20 may move to when unbiased by fluid flow.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from bovine pericardial or other living tissue. Other configurations and/or materials are also contemplated.

In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 can include an outer skirt 24 disposed on and/or extending along an outer surface of the expandable framework 12. In some embodiments, the outer skirt 24 may be disposed at and/or adjacent the lower crown 14. In some embodiments, the outer skirt 24 may be disposed between the expandable framework 12 and the vessel wall in order to prevent fluid, such as blood, flowing around the replacement heart valve implant 10 and/or the expandable framework 12 in a downstream direction. The outer skirt 24 may ensure the fluid flows through the replacement heart valve implant 10 and does not flow around the replacement heart valve implant 10, so as to ensure that the plurality of valve leaflets 20 can stop the flow of fluid when in the closed position.

In some embodiments, the inner skirt 22 may include a polymer, such as a thermoplastic polymer. In some embodiments, the inner skirt 22 may include at least 50 percent by weight of a polymer. In some embodiments, the outer skirt 24 may include a polymer, such as a thermoplastic polymer. In some embodiments, the outer skirt 24 may include at least 50 percent by weight of a polymer. In some embodiments, one or more of the plurality of valve leaflets 20, the inner skirt 22, and/or the outer skirt 24 may be formed of the same polymer or polymers. In some embodiments, the polymer may be a polyurethane. In some embodiments, the inner skirt 22 and/or the outer skirt 24 may be substantially impervious to fluid. In some embodiments, the inner skirt 22 and/or the outer skirt 24 may be formed from a thin tissue (e.g., bovine pericardial, etc.). In some embodiments, the inner skirt 22 and/or the outer skirt 24 may be formed from a coated fabric material. In some embodiments, the inner skirt 22 and/or the outer skirt 24 may be formed from a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of the plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 and/or the plurality of frame struts using one or more methods including but not limited to tying with sutures or filaments, adhesive bonding, melt bonding, embedding or over molding, welding, etc.

In some embodiments, the replacement heart valve implant 10 may include a sealing member disposed on the expandable framework 12 proximate the inflow end. In some embodiments, the sealing member may include and/or may be the inner skirt 22. In some embodiments, the sealing member may include and/or may be the outer skirt 24. In some embodiments, the sealing member may include and/or may be the inner skirt 22 and the outer skirt 24. Other configurations are also contemplated.

In some embodiments, the expandable framework 12 and/or the replacement heart valve implant 10 may have an outer extent of about 23 millimeters (mm), about 25 mm, about 27 mm, about 30 mm, etc. in an unconstrained configuration (e.g., in the radially expanded configuration). In some embodiments, the expandable framework 12 and/or the replacement heart valve implant 10 may have an outer extent of about 10 mm, about 9 mm about 8 mm, about 7 mm, about 6 mm, etc. in the radially collapsed configuration. Other configurations are also contemplated.

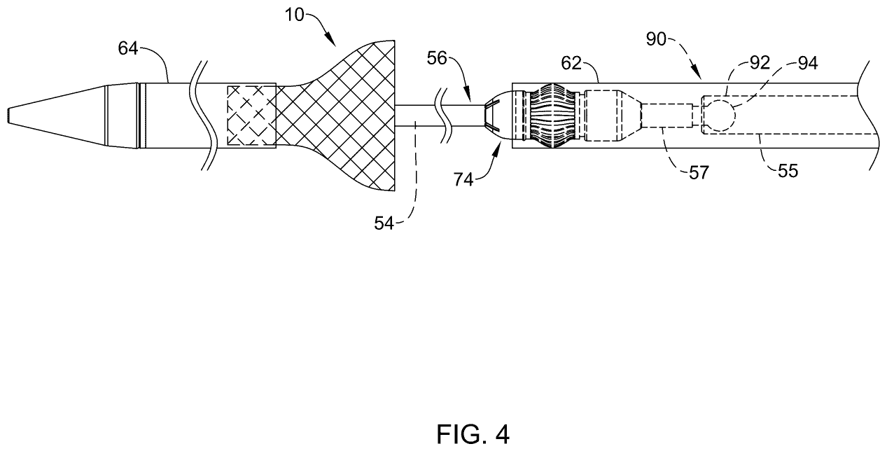

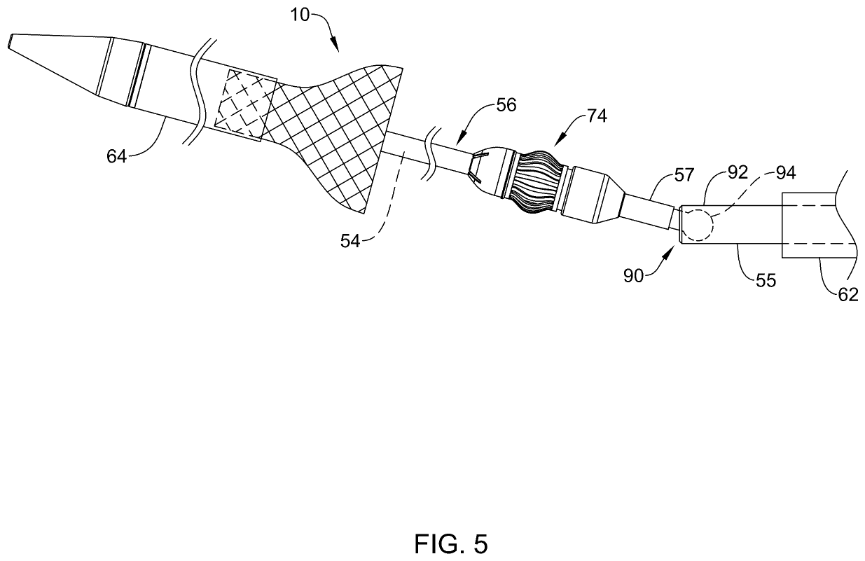

FIG. 2-7 illustrate selected aspects of a replacement heart valve system including the replacement heart valve implant 10 and an implant delivery system 30 for delivering a replacement heart valve implant to a native heart valve (e.g., the aortic valve). The implant delivery system 30 may be compatible with and/or usable with the replacement heart valve implant 10. In FIG. 2, some elements that would be hidden from view are shown in phantom to show relative positioning. In FIG. 2, only the expandable framework 12 of the replacement heart valve implant 10 is shown. Other elements of the replacement heart valve implant 10 have been omitted to improve clarity. It should also be noted that FIG. 2 includes at least one change of scale (e.g., all parts of the figure are not drawn to the same scale) to improve viewability and show additional detail of selected aspects of the implant delivery system 30. In FIGS. 4-5, the replacement heart valve implant 10 is shown schematically to improve clarity.

The implant delivery system 30 may include a handle assembly 40 and an elongate shaft assembly 50 extending distally from the handle assembly 40. The handle assembly 40 may include a first end 41 and a second end 42 opposite the first end 41. The elongate shaft assembly 50 may extend distally from the second end 42 of the handle assembly 40. The handle assembly 40 may include one or more rotatable knobs. In some embodiments, the one or more rotatable knobs may include a first rotatable knob 43 and a second rotatable knob 44. In at least some embodiments, the first rotatable knob 43 and/or the second rotatable knob 44 may be configured to rotate about a central longitudinal axis of the implant delivery system 30 and/or the handle assembly 40.

In some embodiments, a distal portion of the implant delivery system 30 and/or the elongate shaft assembly 50 may include an implant holding portion 60 configured to engage with and/or constrain the replacement heart valve implant 10 and/or the expandable framework 12 in the radially collapsed configuration when the implant holding portion 60 is disposed in a closed configuration, as seen in FIG. 2. In some embodiments, the implant holding portion 60 may comprise a proximal sheath 62 and a distal sheath 64. In some embodiments, the proximal sheath 62 and/or the distal sheath 64 may be formed from a polymeric material. In some embodiments, the proximal sheath 62 and/or the distal sheath 64 may include a reinforcing structure disposed therein and/or thereon. In some embodiments, the reinforcing structure may be a coil, a mesh, one or more filaments, bands, or strips, or another suitable structure. Other configurations are also contemplated.

In some embodiments, the elongate shaft assembly 50 may comprise an outer tubular member 52 coupled to the proximal sheath 62, an intermediate tubular member 56 slidably disposed within the outer tubular member 52, and an inner shaft 54 coupled to the distal sheath 64 and slidably disposed within the intermediate tubular member 56.

In some embodiments, the outer tubular member 52 may be coupled to and/or axially secured to the handle assembly 40 and disposed about the inner shaft 54. In the context of this disclosure, “axially secured” (and/or variants thereof) generally means that the feature or element is not free floating in an axial direction relative to another feature or element. For example, the feature or element may not be permitted to move freely in an axial direction or on its own but in some embodiments may be movable in an axial direction using a means or mechanism for controlled movement. In some embodiments, the inner shaft 54 may be slidably disposed within a lumen of the outer tubular member 52.

In some embodiments, the inner shaft 54 may be coupled to and/or axially secured to the handle assembly 40. In some embodiments, the elongate shaft assembly 50 may comprise a distal tip 58 fixedly secured to a distal end of the inner shaft 54 distal of the implant holding portion 60. In some embodiments, the inner shaft 54 may extend distally from the handle assembly 40 within the outer tubular member 52 to the distal tip 58 disposed distal of the implant holding portion 60.

In some embodiments, the intermediate tubular member 56 may be disposed within and/or radially inward of the outer tubular member 52 and about and/or radially outward of the inner shaft 54. In some embodiments, the intermediate tubular member 56 may be fixedly attached to the handle assembly 40. In some embodiments, the inner shaft 54 may be slidably disposed within a lumen of the outer tubular member 52 and/or the intermediate tubular member 56. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In some embodiments, the proximal sheath 62 may be fixedly attached to the outer tubular member 52. In some embodiments, the proximal sheath 62 may extend distally from a distal end of the outer tubular member 52. In some embodiments, the distal tip 58 may be fixedly attached to the inner shaft 54. In some embodiments, the distal sheath 64 may be fixedly attached to the distal tip 58 and/or the inner shaft 54. In some embodiments, the distal sheath 64 may be fixedly attached to the inner shaft 54 via the distal tip 58 (e.g., the distal sheath 64 is fixedly attached to the distal tip 58 which is fixedly attached to the inner shaft 54). In some embodiments, the distal sheath 64 may extend proximally from the distal tip 58. In some embodiments, the inner shaft 54 may include and/or at least partially define a lumen extending therethrough. In some embodiments, the lumen may extend through the handle assembly 40. In at least some embodiments, the lumen may be configured to slidably receive a guidewire therein.

In some embodiments, the handle assembly 40 may be configured to manipulate and/or translate the proximal sheath 62 and/or the distal sheath 64 relative to each other using the first rotatable knob 43 and/or the second rotatable knob 44. In some embodiments, the handle assembly 40 may be configured to manipulate and/or translate the inner shaft 54 and/or the distal sheath 64 relative to the elongate shaft assembly 50, the outer tubular member 52, the intermediate tubular member 56, and/or the proximal sheath 62. In some embodiments, the handle assembly 40 may be configured to manipulate and/or translate the outer tubular member 52 and/or the proximal sheath 62 relative to the elongate shaft assembly 50, the inner shaft 54, the intermediate tubular member 56, and/or the distal sheath 64. In some embodiments, the handle assembly 40 may be configured to axially move the inner shaft 54 relative to the outer tubular member 52 and/or the intermediate tubular member 56. In some embodiments, the handle assembly 40 may be configured to axially move the outer tubular member 52 relative to the inner shaft 54 and/or the intermediate tubular member 56.

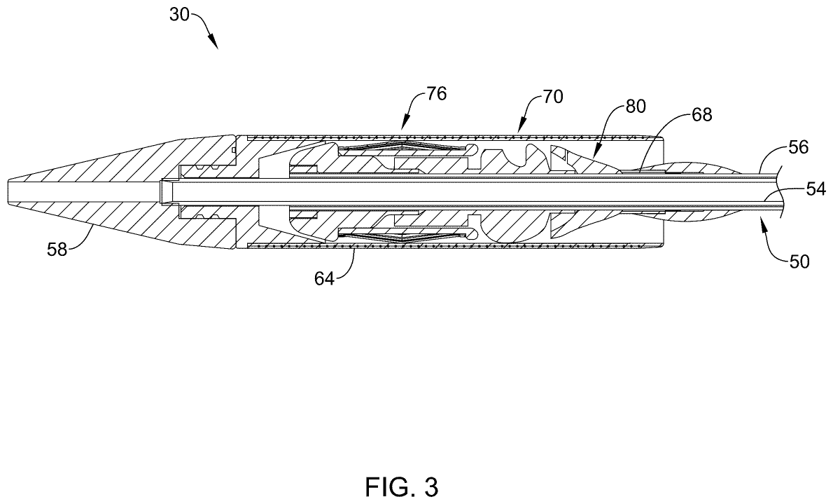

During delivery of the replacement heart valve implant 10 to a treatment site (e.g., the native heart valve, the aortic valve, etc.), the replacement heart valve implant 10 and/or the expandable framework 12 may be disposed at least partially within the proximal sheath 62 and/or the distal sheath 64 in the radially collapsed configuration when the implant holding portion 60 is disposed in a closed configuration (e.g., FIG. 2). In some embodiments, the proximal sheath 62 and/or the distal sheath 64 may collectively define the implant holding portion 60 of the implant delivery system 30. In some embodiments, the implant holding portion 60 may be configured to constrain the replacement heart valve implant 10 and/or the expandable framework 12 in the radially collapsed configuration when the implant holding portion 60 is disposed in the closed configuration (e.g., FIG. 2). In some embodiments, the replacement heart valve implant 10 and/or the expandable framework 12 may be releasably coupled to the inner shaft 54, the intermediate tubular member 56, and/or a stent holder 70 (e.g., FIG. 3; described further below) when the replacement heart valve implant 10 and/or the expandable framework 12 is constrained within the implant holding portion 60 of the implant delivery system 30 in the radially collapsed configuration.

In some embodiments, the implant delivery system 30 and/or the elongate shaft assembly 50 may comprise a proximal shuttle assembly 74 (e.g., FIG. 2) disposed on the elongate shaft assembly 50 and/or the intermediate tubular member 56 proximal of the replacement heart valve implant 10 and/or the expandable framework 12 when the replacement heart valve implant 10 and/or the expandable framework 12 is constrained within the implant holding portion 60 of the implant delivery system 30 in the radially collapsed configuration. In some embodiments, the proximal shuttle assembly 74 may be configured to slide along the elongate shaft assembly 50 and/or the intermediate tubular member 56. In some embodiments, the proximal shuttle assembly 74 may be fixedly attached to the elongate shaft assembly 50 and/or the intermediate tubular member 56. In some embodiments, the proximal shuttle assembly 74 may be configured to center the elongate shaft assembly 50 and/or the intermediate tubular member 56 within the proximal sheath 62 during loading of the replacement heart valve implant 10 and/or the expandable framework 12 into the implant delivery system 30 and/or the implant holding portion 60. In some embodiments, the proximal shuttle assembly 74 may include a plurality of spring elements self-biased to expand radially outward from the elongate shaft assembly 50 and/or the intermediate tubular member 56, thereby exerting a radially outward force against an inner surface of the proximal sheath 62 during loading of the replacement heart valve implant 10 and/or the expandable framework 12 into the implant delivery system 30 and/or the implant holding portion 60 and/or when the implant holding portion 60 is disposed in the closed configuration.

In some embodiments, the proximal sheath 62 may be configured to cover the proximal portion and/or the outflow end of the replacement heart valve implant 10 and/or the expandable framework 12 in the radially collapsed configuration when the implant holding portion 60 is disposed in the closed configuration, and the distal sheath 64 may be configured to cover the distal portion and/or the inflow end of the replacement heart valve implant 10 and/or the expandable framework 12 in the radially collapsed configuration when the implant holding portion 60 is disposed in the closed configuration. In some embodiments, the proximal sheath 62 may be disposed adjacent to the distal sheath 64 in the closed configuration. In some embodiments, the proximal sheath 62 may abut the distal sheath 64 in the closed configuration. In some embodiments, the proximal sheath 62 may be axially spaced apart from the distal sheath 64 in the closed configuration. In some embodiments, the proximal sheath 62 may be axially spaced apart from the distal sheath 64 in the closed configuration by less than 20% of an overall length of the replacement heart valve implant 10 and/or the expandable framework 12. In some embodiments, the proximal sheath 62 may be axially spaced apart from the distal sheath 64 in the closed configuration by less than 15% of an overall length of the replacement heart valve implant 10 and/or the expandable framework 12. In some embodiments, the proximal sheath 62 may be axially spaced apart from the distal sheath 64 in the closed configuration by less than 10% of an overall length of the replacement heart valve implant 10 and/or the expandable framework 12. In some embodiments, the proximal sheath 62 may be axially spaced apart from the distal sheath 64 in the closed configuration by less than 5% of an overall length of the replacement heart valve implant 10 and/or the expandable framework 12. Other configurations are also contemplated.

In some embodiments, the implant holding portion 60 and/or the elongate shaft assembly 50 may include the stent holder 70, seen in FIG. 3. In at least some embodiments, the stent holder 70 may be fixedly attached to the elongate shaft assembly 50. In some embodiments, the stent holder 70 may be fixedly attached to the intermediate tubular member 56 of the elongate shaft assembly 50. In some embodiments, the stent holder 70 may be integrally formed with the elongate shaft assembly 50 and/or the intermediate tubular member 56. In some embodiments, the stent holder 70 may be configured to engage the expandable framework 12 in the radially collapsed configuration and/or when the replacement heart valve implant 10 is constrained within the implant holding portion 60 of the implant delivery system 30. In some embodiments, the stent holder 70 may include at least one projection configured to engage the expandable framework 12 in the radially collapsed configuration. In some embodiments, the at least one projection may be configured to engage the inflow end of the expandable framework 12 in the radially collapsed configuration. In some embodiments, the at least one projection may extend into and/or through interstices of the expandable framework 12. In some embodiments, the expandable framework 12 may include at least one mounting loop configured to receive and/or engage with the at least one projection. Other configurations are also contemplated.

In some embodiments, the implant delivery system 30 and/or the elongate shaft assembly 50 may include the stent holder 70 configured to engage the expandable framework 12 of the replacement heart valve implant 10 in the radially collapsed configuration and/or when the replacement heart valve implant 10 is constrained within the implant holding portion 60 of the implant delivery system 30. In some embodiments, the stent holder 70 may include a body, a first end portion extending proximally from the body, and a second end portion disposed opposite the first end portion. In some embodiments, at least a portion of the first end portion may extend radially outward from and/or radially outward of the body. In some embodiments, the first end portion may have a generally bulbous shape. In some embodiments, the stent holder 70 may be configured and/or adapted to be visible under fluoroscopy. In some embodiments, the stent holder 70 may be formed from stainless steel. Some suitable but non-limiting materials for the stent holder 70 and/or components or elements thereof are described below.

In some embodiments, the stent holder 70 may include a lumen extending longitudinally and/or axially therethrough. In at least some embodiments, at least a portion of the elongate shaft assembly 50 may extend longitudinally and/or axially through the lumen of the stent holder 70.

In some embodiments, the implant delivery system 30 and/or the elongate shaft assembly 50 may include a primary visual indicator 68 (e.g., FIG. 3) disposed within the replacement heart valve implant 10 when the replacement heart valve implant 10 and/or the expandable framework 12 is constrained within the implant holding portion 60 in the radially collapsed configuration. The primary visual indicator 68 may be configured and/or adapted to be visible under fluoroscopy with an imaging device. Other imaging means suitable for use with transcatheter surgical procedures are also contemplated. The implant delivery system 30 and/or the primary visual indicator 68 may be configured to cooperate with the imaging device to position the replacement heart valve implant 10 at a desired insertion depth within the native heart valve (e.g., the aortic valve). In some embodiments, the primary visual indicator 68 may be fixedly attached to the elongate shaft assembly 50 and/or the intermediate tubular member 56 by a shrink wrap or by an adhesive element. In some embodiments, the primary visual indicator 68 may be and/or may include a marker band. In some embodiments, the primary visual indicator 68 may be at least partially radiopaque. In some embodiments, the primary visual indicator 68 may be completely radiopaque. Other configurations are also contemplated.

In some embodiments, the implant delivery system 30 and/or the elongate shaft assembly 50 may comprise a distal shuttle assembly 76 (e.g., FIG. 3) disposed on the elongate shaft assembly 50 and/or the intermediate tubular member 56 distal of the replacement heart valve implant 10 and/or the expandable framework 12 when the replacement heart valve implant 10 and/or the expandable framework 12 is constrained within the implant holding portion 60 of the implant delivery system 30 in the radially collapsed configuration. In some embodiments, the distal shuttle assembly 76 may be configured to slide along the elongate shaft assembly 50 and/or the intermediate tubular member 56. In some embodiments, the distal shuttle assembly 76 may be fixedly attached to the elongate shaft assembly 50 and/or the intermediate tubular member 56. In some embodiments, the distal shuttle assembly 76 may be configured to center the elongate shaft assembly 50 and/or the intermediate tubular member 56 within the distal sheath 64 during loading of the replacement heart valve implant 10 and/or the expandable framework 12 into the implant delivery system 30 and/or the implant holding portion 60. In some embodiments, the distal shuttle assembly 76 may include a plurality of spring elements self-biased to expand radially outward from the elongate shaft assembly 50 and/or the intermediate tubular member 56, thereby exerting a radially outward force against an inner surface of the distal sheath 64 during loading of the replacement heart valve implant 10 and/or the expandable framework 12 into the implant delivery system 30 and/or the implant holding portion 60 and/or when the implant holding portion 60 is disposed in the closed configuration. In some embodiments, a proximal portion of the distal shuttle assembly 76 may be engaged with a distal portion of the stent holder 70. In some embodiments, the proximal portion of the distal shuttle assembly 76 may be disposed radially outward of and/or may surround the distal portion of the stent holder 70. Other configurations are also contemplated.

In some embodiments, the implant delivery system 30 and/or the implant holding portion 60 may include an atraumatic transition shield 80 (e.g., FIG. 3). The atraumatic transition shield 80 may be disposed adjacent the stent holder 70. In some embodiments, the atraumatic transition shield 80 may be disposed between the stent holder 70 and the handle assembly 40. In some embodiments, the atraumatic transition shield 80 may be disposed proximal of the stent holder 70. In some embodiments, the atraumatic transition shield 80 may be disposed at and/or adjacent the first end portion of the stent holder 70. In some embodiments, the atraumatic transition shield 80 may axially overlap the first end portion of the stent holder 70. In some embodiments, the atraumatic transition shield 80 may be disposed radially outward of at least a portion of the first end portion of the stent holder 70. In some embodiments, the atraumatic transition shield 80 may be tapered radially inward in the downstream direction and/or the proximal direction and/or toward the handle assembly 40. The atraumatic transition shield 80 may be configured to prevent the replacement heart valve implant 10, the expandable framework 12, the plurality of valve leaflets 20, etc. from catching on the stent holder 70 as the implant delivery system 30 is withdrawn after deploying the replacement heart valve implant 10.

In some embodiments, the primary visual indicator 68 may be disposed adjacent a proximal end of the atraumatic transition shield 80. In some embodiments, the primary visual indicator 68 may be disposed downstream and/or proximal of the atraumatic transition shield 80. In some embodiments, the primary visual indicator 68 and the atraumatic transition shield 80 may axially overlap. In some embodiments, the primary visual indicator 68 may be fixedly attached to the elongate shaft assembly 50. In some embodiments, the primary visual indicator 68 may be embedded in the elongate shaft assembly 50 and/or the intermediate tubular member 56. In some embodiments, the primary visual indicator 68 may be secured and/or fixedly attached to the intermediate tubular member 56, for example by adhesive bonding, welding, shrink wrap, etc. Other configurations are also contemplated.

FIGS. 4-7 illustrate selected aspects of the implant delivery system 30 and/or the elongate shaft assembly 50. In some embodiments, the elongate shaft assembly 50 and/or the intermediate tubular member 56 may comprise a joint 90. In at least some embodiments, the joint 90 may be disposed within the implant holding portion 60. In some embodiments, the joint 90 may be disposed within the implant holding portion 60 in the closed configuration. In some embodiments, the joint 90 may be disposed within the implant holding portion 60 in the release configuration. Other configurations are also contemplated.

In some embodiments, the joint 90 may be configured to movably couple a distal portion 57 of the intermediate tubular member 56 to a proximal portion 55 of the intermediate tubular member 56. In some embodiments, the joint 90 may be fixedly attached to the proximal portion 55 of the intermediate tubular member 56 and/or the distal portion 57 of the intermediate tubular member 56. In some embodiments, the joint 90 may be integrally formed with and/or may be monolithically formed within the proximal portion 55 of the intermediate tubular member 56 and/or the distal portion 57 of the intermediate tubular member 56. In some embodiments, the joint 90 may be manufactured separately from the proximal portion 55 of the intermediate tubular member 56 and/or the distal portion 57 of the intermediate tubular member 56 and subsequently attached thereto. Other configurations are also contemplated.

In some embodiments, the joint 90 may comprise a ball joint having a socket 92 fixedly attached to the proximal portion 55 of the intermediate tubular member 56 immediately adjacent the joint 90 and a ball 94 fixedly attached to the distal portion 57 of the intermediate tubular member 56 immediately adjacent the joint 90, as seen in FIGS. 4-5. In some embodiments, the socket 92 may be fixedly attached to the distal portion 57 of the intermediate tubular member 56 immediately adjacent the joint 90 and the ball 94 may be fixedly attached to the proximal portion 55 of the intermediate tubular member 56 immediately adjacent the joint 90. The socket 92 may be configured to pivotably receive the ball 94 therein. The joint 90 and/or the ball joint may be configured to permit the distal portion 57 of the intermediate tubular member 56 immediately adjacent the joint 90 to bend and/or deflect laterally relative to the proximal portion 55 of the intermediate tubular member 56 immediately adjacent the joint 90 when the distal portion 57 of the intermediate tubular member 56 immediately adjacent the joint 90 is unconstrained by the proximal sheath 62. In at least some embodiments, the socket 92 and the ball 94 may be permanently coupled together and/or may be prevented from detaching from each other. Other configurations are also contemplated.

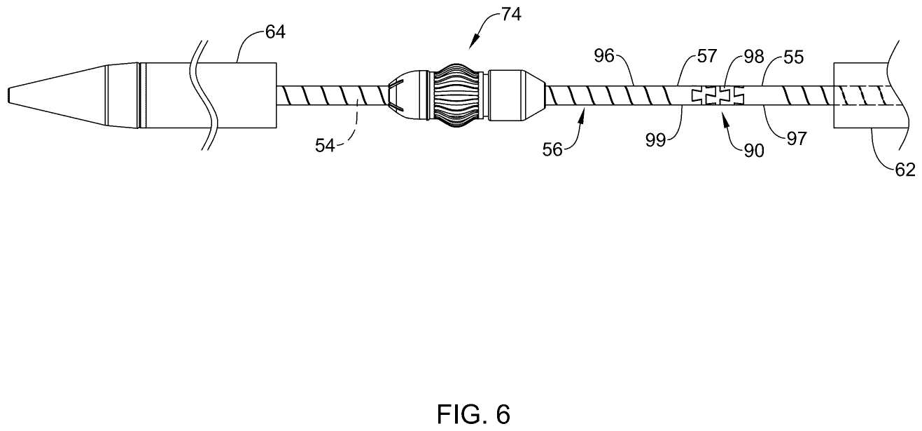

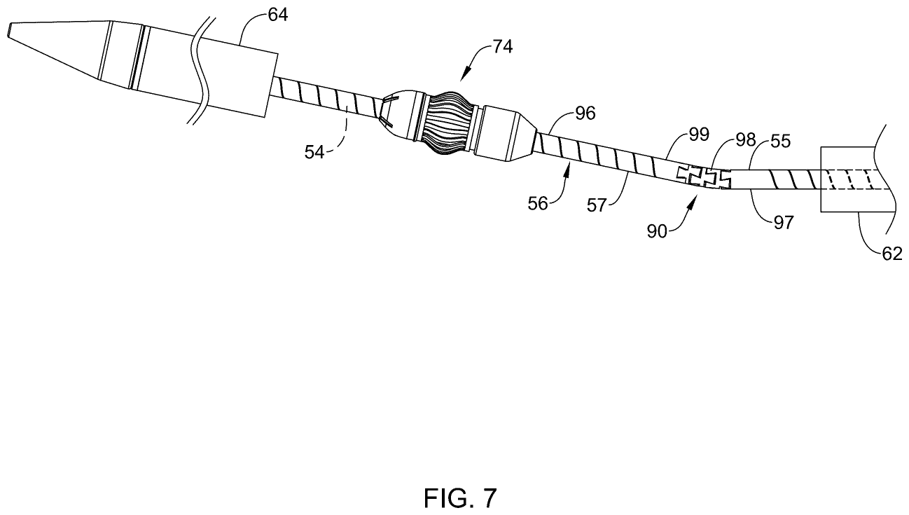

In some embodiments, the joint 90 may comprise a metallic tubular member 96 extending between the proximal portion 55 of the intermediate tubular member 56 immediately adjacent the joint 90 and the distal portion 57 of the intermediate tubular member 56 immediately adjacent the joint 90, as seen in FIGS. 6-7. In FIGS. 6-7, the replacement heart valve implant 10 and/or the expandable framework 12 is omitted to improve clarity. In some embodiments, the metallic tubular member 96 may be fixedly attached to the distal portion 57 of the intermediate tubular member 56 immediately adjacent the joint 90 and the proximal portion 55 of the intermediate tubular member 56 immediately adjacent the joint 90. In some embodiments, the metallic tubular member 96 may comprise a hypotube or other tubular member. In some embodiments, the metallic tubular member 96 may comprise a plurality of interconnected links 98. In some embodiments, the plurality of interconnected links 98 may be cut from a monolithic tube. The plurality of interconnected links 98 may be configured to move relative to each other to permit the metallic tubular member 96 to bend and/or deflect laterally at the joint 90 without decoupling from each other. The plurality of interconnected links 98 may be configured to move relative to each other to permit the metallic tubular member 96 to bend and/or deflect laterally such that the proximal portion 55 of the intermediate tubular member 56 and the distal portion 57 of the intermediate tubular member 56 bend and/or deflect laterally relative to each other at the joint 90 without decoupling from each other. Other configurations are also contemplated. In some embodiments, a proximal portion 97 of the metallic tubular member 96 disposed proximal of the plurality of interconnected links 98 may be configured to bend and/or deflect laterally relative to a distal portion 99 of the metallic tubular member 96 disposed distal of the plurality of interconnected links 98 at the plurality of interconnected links 98. In some embodiments, the proximal portion 97 of the metallic tubular member 96 disposed proximal of the plurality of interconnected links 98 may be fixedly attached to and/or integrally formed with the proximal portion 55 of the intermediate tubular member 56. In some embodiments, the distal portion 99 of the metallic tubular member 96 disposed distal of the plurality of interconnected links 98 may be fixedly attached to and/or integrally formed with the distal portion 57 of the intermediate tubular member 56. In some embodiments, the proximal portion 97 of the metallic tubular member 96 disposed proximal of the plurality of interconnected links 98 and/or the distal portion 99 of the metallic tubular member 96 disposed distal of the plurality of interconnected links 98 may comprise a helical cut and/or other features configured to improve flexibility and/or navigability of the elongate shaft assembly 50. Other configurations are also contemplated.

In some embodiments, the inner shaft 54 may comprise a lumen extending therethrough, as seen in FIG. 3 for example. In at least some embodiments, the lumen may be configured to slidably receive a guidewire therein. In some embodiments, the inner shaft 54 may extend through the joint 90. In some embodiments, the inner shaft 54 may be configured to prevent the socket 92 and the ball 94 from detaching from each other. In some embodiments, the inner shaft 54 may be configured to prevent the plurality of interconnected links 98 from decoupling from each other. Other configurations are also contemplated.

In some embodiments, the joint 90 may be configured to permit at least one degree of movement between the distal portion 57 of the intermediate tubular member 56 immediately adjacent the joint 90 and the proximal portion 55 of the intermediate tubular member 56 immediately adjacent the joint 90 when the distal portion 57 of the intermediate tubular member 56 immediately adjacent the joint 90 is laterally unconstrained relative to the proximal portion 55 of the intermediate tubular member 56 immediately adjacent the joint 90. In some embodiments, the joint 90 may be configured to permit at least one degree of movement between the distal portion 57 of the intermediate tubular member 56 immediately adjacent the joint 90 and the proximal portion 55 of the intermediate tubular member 56 immediately adjacent the joint 90 when the distal portion 57 of the intermediate tubular member 56 immediately adjacent the joint 90 is laterally unconstrained relative to the proximal portion 55 of the intermediate tubular member 56 immediately adjacent the joint 90 by the outer tubular member 52 and/or the proximal sheath 62.

In some embodiments, the proximal sheath 62 may be configured to shift between a first position (e.g., the closed configuration) in which the proximal sheath 62 laterally constrains the distal portion 57 of the intermediate tubular member 56 immediately adjacent the joint 90 relative to the proximal portion 55 of the intermediate tubular member 56 immediately adjacent the joint 90, and a second position (e.g., a release configuration) in which the proximal sheath 62 does not laterally constrain the distal portion 57 of the intermediate tubular member 56 immediately adjacent the joint 90 relative to the proximal portion 55 of the intermediate tubular member 56 immediately adjacent the joint 90.

In at least some embodiments, the proximal shuttle assembly 74 may be configured to engage an inner surface of the proximal sheath 62 in the first position (e.g., the closed configuration) to laterally constrain the distal portion 57 of the intermediate tubular member 56 immediately adjacent the joint 90 relative to the proximal portion 55 of the intermediate tubular member 56 immediately adjacent the joint 90, as seen in FIGS. 4 and 6 for example. In some embodiments, engagement of the proximal shuttle assembly 74 with the inner surface of the proximal sheath 62 may substantially prevent deflection of the distal portion 57 of the intermediate tubular member 56 immediately adjacent the joint 90 relative to the proximal portion 55 of the intermediate tubular member 56 immediately adjacent the joint 90.

In some embodiments, when the proximal sheath 62 is disposed in the second position (e.g., the release configuration), the proximal shuttle assembly 74 may be disengaged from and/or disposed distal of the proximal sheath 62 such that the distal portion 57 of the intermediate tubular member 56 immediately adjacent the joint 90 is laterally unconstrained relative to the proximal portion 55 of the intermediate tubular member 56 immediately adjacent the joint 90, as seen in FIGS. 5 and 7 for example.

In some embodiments, the joint 90 may be configured to permit the distal portion 57 of the intermediate tubular member 56 immediately adjacent the joint 90 to form an oblique angle with the proximal portion 55 of the intermediate tubular member 56 immediately adjacent the joint 90 when the distal portion 57 of the intermediate tubular member 56 immediately adjacent the joint 90 is laterally unconstrained relative to the proximal portion 55 of the intermediate tubular member 56 immediately adjacent the joint 90. In some embodiments, the joint 90 may be configured to permit the distal portion 57 of the intermediate tubular member 56 immediately adjacent the joint 90 to form an obtuse angle with the proximal portion 55 of the intermediate tubular member 56 immediately adjacent the joint 90 when the distal portion 57 of the intermediate tubular member 56 immediately adjacent the joint 90 is laterally unconstrained relative to the proximal portion 55 of the intermediate tubular member 56 immediately adjacent the joint 90.

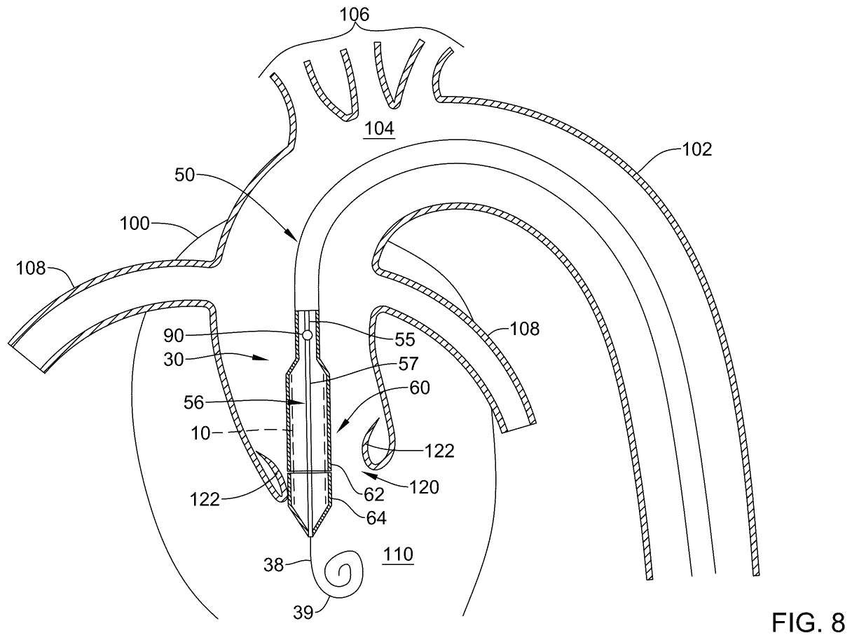

A replacement heart valve system in accordance with the disclosure may comprise the replacement heart valve implant 10, a guidewire 38, and the implant delivery system 30. The guidewire 38 may be slidably disposable within a lumen of the elongate shaft assembly 50 and/or the lumen of the inner shaft 54. Accordingly, the implant delivery system 30 may be configured to axially translate relative to and/or along the guidewire 38 in vivo. In some embodiments, the guidewire 38 may comprise a self-biased distal portion 39 forming a spiral configuration when unconstrained (e.g., which disposed outside of the elongate shaft assembly 50 and/or the inner shaft 54). At least some details related to the replacement heart valve system are illustrated in FIGS. 8-11, discussed further below.

In some embodiments, when the self-biased distal portion 39 of the guidewire 38 is disposed within the lumen of the elongate shaft assembly 50 and/or within the lumen of the inner shaft 54, and the distal portion 57 of the intermediate tubular member 56 immediately adjacent the joint 90 is unconstrained relative to the proximal portion 55 of the intermediate tubular member 56 immediately adjacent the joint 90 (e.g., by the proximal sheath 62), the distal portion 57 of the intermediate tubular member 56 immediately adjacent the joint 90 may be biased laterally by the self-biased distal portion 39 of the guidewire 38.

For reference, FIGS. 8-10 and 12 illustrate schematic partial cut-away views of a portion of a patient's heart 100 including the aortic valve 120 having native valve leaflets 122 disposed within and/or extending from the native valve annulus, a left ventricle 110, and certain connected vasculature, such as the aorta 102 connected to the aortic valve 120 of the patient's heart 100 by the aortic arch 104, the coronary arteries 108, the ostia of the coronary arteries 108, and other large arteries 106 (e.g., subclavian arteries, carotid arteries, brachiocephalic artery) that extend from the aortic arch 104 to important internal organs. For the purpose of this disclosure, the discussion herein is directed toward use in treating the aortic valve 120 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure. Additionally, the views include at least one change of scale.

FIGS. 8-12 schematically illustrate selected aspects of a method of deploying the replacement heart valve implant 10 within a native heart valve (e.g., the aortic valve 120). The method of deploying the replacement heart valve implant 10 within the native heart valve (e.g., the aortic valve 120) may comprise advancing replacement heart valve system and/or the implant delivery system 30 to a position adjacent the native heart valve (e.g., the aortic valve 120). In one example, the replacement heart valve system and/or the implant delivery system 30 may be advanced through the vasculature and across the aortic arch 104 to a position adjacent the native heart valve (e.g., the aortic valve 120), as seen in FIG. 8. Alternative approaches to treat a defective aortic valve and/or other heart valve(s) are also contemplated with the implant delivery system 30. As discussed herein, the replacement heart valve implant 10 may be constrained within the implant holding portion 60 of the implant delivery system 30 in the radially collapsed configuration when the implant delivery system 30 is in the closed configuration (e.g., when the proximal sheath 62 is disposed in a first position and the distal sheath 64 is disposed in a first position).

In some embodiments, the method may comprise positioning a guidewire 38 comprising a self-biased distal portion 39 forming a spiral configuration when unconstrained such that the self-biased distal portion 39 is disposed within the left ventricle 110 of the patient's heart 100. In some embodiments, the method may comprise advancing the implant delivery system 30 to a position adjacent the native heart valve (e.g., the aortic valve 120) over and/or along the guidewire 38 previously positioned within the native heart valve (e.g., the aortic valve 120). In some embodiments, the guidewire 38 may be positioned within the native heart valve (e.g., the aortic valve 120) using a catheter or a tubular member which is then removed prior to advancing the implant delivery system 30 over the guidewire 38, thereby permitting the self-biased distal portion 39 to assume the spiral configuration within the left ventricle 110 of the patient's heart 100. Thereafter, the implant delivery system 30 may be advanced over and/or along the guidewire 38 to the position adjacent the native heart valve (e.g., the aortic valve 120), wherein the guidewire 38 is disposed within the lumen of the inner shaft 54 of the elongate shaft assembly 50 of the implant delivery system 30. Other configurations are also contemplated.

In some embodiments, positioning the replacement heart valve implant 10 and/or the expandable framework 12 within the native heart valve (e.g., the aortic valve 120) may be accomplished by locating the primary visual indicator 68 relative to the native heart valve (e.g., the aortic valve 120). During visualization, the native heart valve (e.g., the aortic valve 120) may be identified and/or visualized under fluoroscopy using known means and/or methods, such as contrast injection. Other configurations are also contemplated.

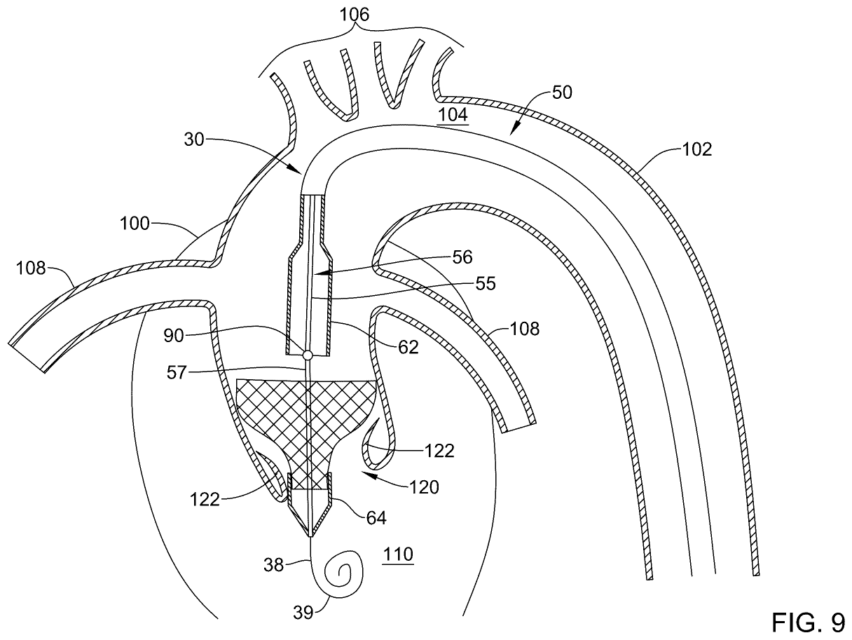

As may be seen in FIG. 8, properties of the implant delivery system 30 and the anatomy of the patient may cause and/or result in the implant delivery system 30 and/or the implant holding portion 60 being biased toward one side of the native heart valve (e.g., the aortic valve 120) and/or away from the aorta 102 within the native heart valve (e.g., the aortic valve 120). In some embodiments, the implant holding portion 60 may be misaligned with the native heart valve (e.g., the aortic valve 120) and/or may be non-coaxial with a center of the native heart valve (e.g., the aortic valve 120) and/or the native valve annulus. Such an arrangement may cause the replacement heart valve implant 10 to be improperly deployed with respect to the native heart valve (e.g., the aortic valve 120), as seen in FIG. 9, which may result in paravalvular leakage, improper function of the replacement heart valve implant 10, and/or other complications. Proper alignment of the replacement heart valve implant 10 with the native heart valve (e.g., the aortic valve 120) and/or the center of the native valve annulus is important for long term success of the procedure and patient health.

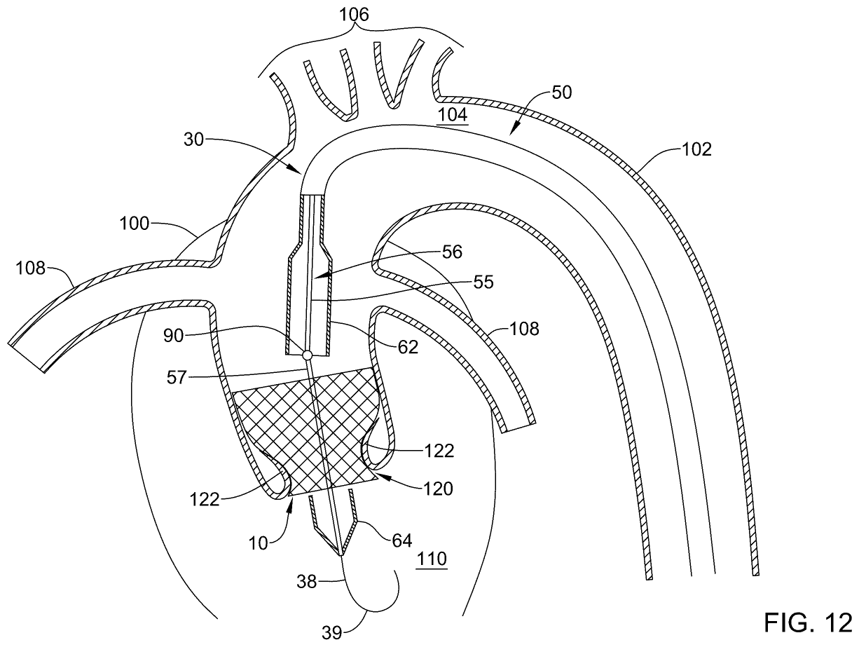

In some embodiments, the method of deploying the replacement heart valve implant 10 and/or the expandable framework 12 within the native heart valve (e.g., the aortic valve 120) may comprise shifting the proximal sheath 62 and the distal sheath 64 of the implant holding portion 60 from the closed configuration (e.g., FIG. 2; FIG. 8) to a release configuration (e.g., FIG. 12) to deploy the replacement heart valve implant 10 and/or the expandable framework 12. In some embodiments, shifting the proximal sheath 62 and the distal sheath 64 of the implant holding portion 60 from the closed configuration to the release configuration may include shifting the proximal sheath 62 and the distal sheath 64 of the implant holding portion 60 axially apart from each other. In some embodiments, the proximal sheath 62 and the distal sheath 64 may be configured to move axially away from each other to deploy the replacement heart valve implant 10 and/or the expandable framework 12.

In some embodiments, the replacement heart valve implant 10 and/or the expandable framework 12 may be deployed and/or released in phases. In some embodiments, in a first phase, the proximal portion of the replacement heart valve implant 10 and/or the expandable framework 12 may be deployed and/or released within and/or adjacent to the native heart valve (e.g., the aortic valve 120). In some embodiments, in the first phase, the proximal portion of the replacement heart valve implant 10 and/or the expandable framework 12 may be deployed and/or released by proximally retracting and/or withdrawing the proximal sheath 62 and/or the outer tubular member 52 relative to the intermediate tubular member 56 and/or the handle assembly 40, as seen in FIGS. 4 and 9 for example. In some embodiments, the method may comprise shifting the proximal sheath 62 from a first position (e.g., FIG. 8) to a second position (e.g., FIG. 9), thereby releasing the proximal portion of the replacement heart valve implant 10 and/or the expandable framework 12 within and/or adjacent to the native heart valve (e.g., the aortic valve 120). Upon release of the proximal portion of the replacement heart valve implant 10 and/or the expandable framework 12, the proximal portion of the replacement heart valve implant 10 and/or the expandable framework 12 may be configured to shift toward the radially expanded configuration. As seen in FIG. 9, if the implant holding portion 60 of the implant delivery system 30 is misaligned with the native heart valve (e.g., the aortic valve 120) and/or the center of the native valve annulus, the replacement heart valve implant 10 and/or the expandable framework 12 may fail to properly engage the native heart valve (e.g., the aortic valve 120) when released.

In some embodiments, after shifting the proximal sheath 62 to the release configuration and/or the second position, the practitioner may check the position of and/or may check the alignment of the replacement heart valve implant 10 and/or the expandable framework 12 within the native heart valve (e.g., the aortic valve 120) and/or the native valve annulus. If necessary, the replacement heart valve implant 10 and/or the expandable framework 12 may be recaptured and/or repositioned to improve the position and/or alignment of the replacement heart valve implant 10 and/or the expandable framework 12 within the native heart valve (e.g., the aortic valve 120) and/or the native valve annulus.

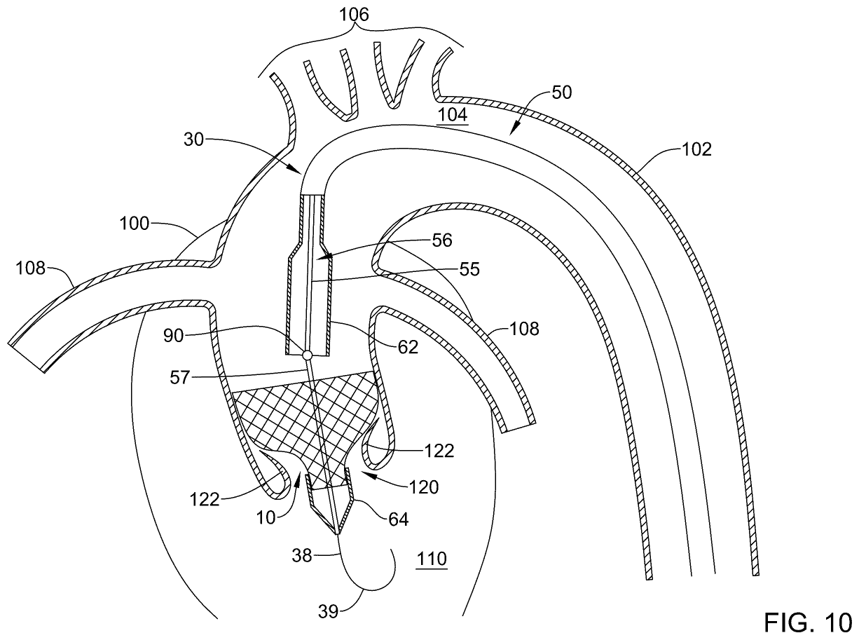

In some embodiments, the method of deploying the replacement heart valve implant 10 and/or the expandable framework 12 may comprise deflecting the distal portion 57 of the intermediate tubular member 56 relative to the proximal portion 55 of the intermediate tubular member 56 at the joint 90, as seen in FIG. 10. In some embodiments, the method of deploying the replacement heart valve implant 10 and/or the expandable framework 12 may comprise, after shifting the proximal sheath 62 from the first position to the second position, withdrawing at least a portion of the self-biased distal portion 39 of the guidewire 38 into the lumen of the inner shaft 54, as seen in FIG. 10. In some embodiments, the self-biased distal portion 39 of the guidewire 38 may be configured to deflect the distal portion 57 of the intermediate tubular member 56 laterally relative to the proximal portion 55 of the intermediate tubular member 56. In some embodiments, the self-biased distal portion 39 of the guidewire 38 may be configured to deflect the distal portion 57 of the intermediate tubular member 56 laterally relative to the proximal portion 55 of the intermediate tubular member 56 at the joint 90. In some embodiments, the self-biased distal portion 39 of the guidewire 38 may be configured to apply and/or may apply a biasing force against the inner shaft 54 and/or the distal portion 57 of the intermediate tubular member 56 when at least a portion of the self-biased distal portion 39 of the guidewire 38 is constrained within the lumen of the inner shaft 54.

In some embodiments, the distal portion 57 of the intermediate tubular member 56 may be substantially prevented from deflecting relative to the proximal portion 55 of the intermediate tubular member 56 at the joint 90 when the proximal sheath 62 is disposed in the first position. In some embodiments, the distal portion 57 of the intermediate tubular member 56 may be permitted to deflect relative to the proximal portion 55 of the intermediate tubular member 56 at the joint 90 when the proximal sheath 62 is disposed in the second position. In some embodiments, the distal portion 57 of the intermediate tubular member 56 may be substantially prevented from deflecting relative to the proximal portion 55 of the intermediate tubular member 56 at the joint 90 when the proximal sheath 62 is disposed in the closed configuration. In some embodiments, the distal portion 57 of the intermediate tubular member 56 may be permitted to deflect relative to the proximal portion 55 of the intermediate tubular member 56 at the joint 90 when the proximal sheath 62 is disposed in the release configuration.

In some embodiments, the distal portion 57 of the intermediate tubular member 56 immediately adjacent the joint 90 may be substantially prevented from deflecting relative to the proximal portion 55 of the intermediate tubular member 56 immediately adjacent the joint 90 when the proximal sheath 62 is disposed in the first position. In some embodiments, the distal portion 57 of the intermediate tubular member 56 immediately adjacent the joint 90 may be permitted to deflect relative to the proximal portion 55 of the intermediate tubular member 56 immediately adjacent the joint 90 when the proximal sheath 62 is disposed in the second position. In some embodiments, the distal portion 57 of the intermediate tubular member 56 immediately adjacent the joint 90 may be substantially prevented from deflecting relative to the proximal portion 55 of the intermediate tubular member 56 immediately adjacent the joint 90 when the proximal sheath 62 is disposed in the closed configuration. In some embodiments, the distal portion 57 of the intermediate tubular member 56 immediately adjacent the joint 90 may be permitted to deflect relative to the proximal portion 55 of the intermediate tubular member 56 immediately adjacent the joint 90 when the proximal sheath 62 is disposed in the release configuration.

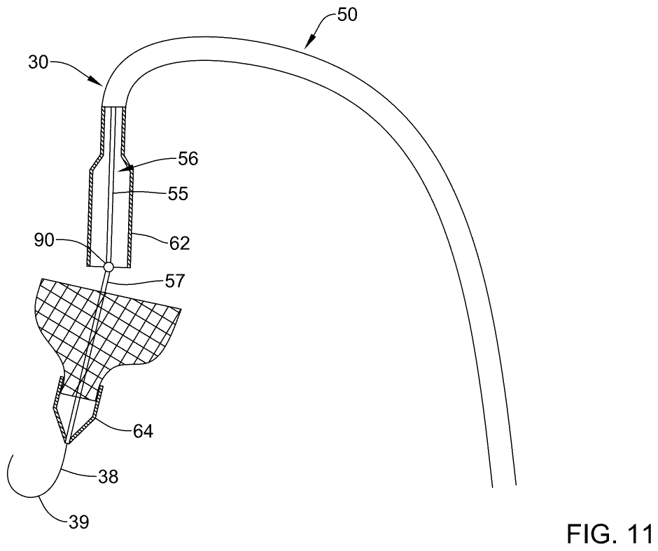

In some embodiments, rotation of the guidewire 38 relative to the implant delivery system 30, the elongate shaft assembly 50, the inner shaft 54, the intermediate tubular member 56, etc. may change a direction of deflection of the distal portion 57 of the intermediate tubular member 56 relative to the proximal portion 55 of the intermediate tubular member 56 at the joint 90. For example, rotation of the guidewire 38 about 180 degrees may cause the distal portion 57 of the intermediate tubular member 56 to deflect in an opposite direction at the joint 90, as seen schematically in FIG. 11. In some embodiments, rotation of the guidewire 38 relative to the implant delivery system 30, the elongate shaft assembly 50, the inner shaft 54, the intermediate tubular member 56, etc. may change a direction of the biasing force applied by the guidewire 38 to the inner shaft 54 and/or the distal portion 57 of the intermediate tubular member 56 when the self-biased distal portion 39 of the guidewire 38 is constrained within the lumen of the inner shaft 54. The change in direction of the biasing force may cause and/or result in a commensurate change in the direction of deflection of the distal portion 57 of the intermediate tubular member 56 relative to the proximal portion 55 of the intermediate tubular member 56 at the joint 90.

In some embodiments, a length of the at least a portion of the self-biased distal portion 39 withdrawn into the lumen of the inner shaft 54 may influence and/or affect the biasing force applied to the inner shaft 54 and/or the distal portion 57 of the intermediate tubular member 56 when the self-biased distal portion 39 of the guidewire 38 is constrained within the lumen of the inner shaft 54. In some embodiments, a shorter length of the at least a portion of the self-biased distal portion 39 of the guidewire 38 constrained within the lumen of the inner shaft 54 may exert less biasing force against the inner shaft 54 and/or the distal portion 57 of the intermediate tubular member 56 than a longer length of the at least a portion of the self-biased distal portion 39 of the guidewire 38 constrained within the lumen of the inner shaft 54. Conversely, a longer length of the at least a portion of the self-biased distal portion 39 of the guidewire 38 constrained within the lumen of the inner shaft 54 may exert more biasing force against the inner shaft 54 and/or the distal portion 57 of the intermediate tubular member 56 than a shorter length of the at least a portion of the self-biased distal portion 39 of the guidewire 38 constrained within the lumen of the inner shaft 54 a shorter length of the at least a portion of the self-biased distal portion 39 of the guidewire 38 constrained within the lumen of the inner shaft 54. Other configurations are also contemplated.

As a result of these properties, the user may use movement of the guidewire 38 relative to the implant delivery system 30 to manipulate the positioning and/or orientation of the implant delivery system 30, the implant holding portion 60 (and/or portions thereof), the distal portion 57 of the intermediate tubular member 56, and/or the replacement heart valve implant 10 within the native heart valve (e.g., the aortic valve 120) to improve alignment of the replacement heart valve implant 10 with the native heart valve (e.g., the aortic valve 120) and/or the center of the native valve annulus.

In some embodiments, in a second phase of the deployment process and/or method, the distal portion of the replacement heart valve implant 10 and/or the expandable framework 12 may be deployed and/or released. In some embodiments, in the second phase, the distal portion of the replacement heart valve implant 10 and/or the expandable framework 12 may be deployed and/or released by distally extending the distal sheath 64 and/or the inner shaft 54 relative to the intermediate tubular member 56 and/or the handle assembly 40, as seen in FIG. 12 for example. In some embodiments, after shifting the distal sheath 64 from the closed configuration (e.g., a first position) to the release configuration (e.g., a second position), the replacement heart valve implant 10 and/or the expandable framework 12 may be deployed and/or released within the native heart valve. In some embodiments, the replacement heart valve implant 10 and/or the expandable framework 12 may be configured to be recaptured and/or removed from the native heart valve if deployment is unsuccessful and/or if a problem is observed/encountered.

In some embodiments, in the release configuration, a distal end of the proximal sheath 62 may be axially spaced apart from a proximal end of the distal sheath 64 by a greater distance than in the closed configuration. In some embodiments, in the release configuration, the distal end of the proximal sheath 62 is axially spaced apart from the proximal end of the distal sheath 64 by at least an overall length of the replacement heart valve implant 10.

In some embodiments, the method of deploying the replacement heart valve implant 10 and/or the expandable framework 12 within the native heart valve (e.g., the aortic valve 120) may comprise, after deflecting the distal portion 57 of the intermediate tubular member 56 relative to the proximal portion 55 of the intermediate tubular member 56 at the joint 90, shifting the distal sheath 64 from the first position to the second position, thereby releasing and/or deploying the distal portion of the replacement heart valve implant 10 and/or the expandable framework 12 within the native heart valve (e.g., the aortic valve 120), as seen in FIG. 12.

In some embodiments, after deploying the replacement heart valve implant 10 and/or the expandable framework 12 within the native heart valve (e.g., the aortic valve 120), the method may comprise further retraction and/or withdrawal of the implant delivery system 30 relative to the replacement heart valve implant 10 and/or the expandable framework 12. In some embodiments, after deploying the replacement heart valve implant 10 and/or the expandable framework 12 within the native heart valve (e.g., the aortic valve 120), the method may comprise retraction and/or withdrawal of the implant delivery system 30 from the treatment site, from the position adjacent the native heart valve (e.g., the aortic valve 120), and/or from the patient.

In at least some interventions, the replacement heart valve implant 10 may be deployed within the native heart valve (e.g., the native heart valve is left in place and not excised). Alternatively, the native heart valve (e.g., the aortic valve 120) may be removed (such as through valvuloplasty, for example) and the replacement heart valve implant 10 may be deployed in its place as a replacement.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the expandable framework, the plurality of valve leaflets, the implant delivery system, the handle assembly, the elongate shaft assembly, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An implant delivery system for delivering a replacement heart valve implant to a native heart valve, comprising:
an elongate shaft assembly including an implant holding portion comprising a proximal sheath and a distal sheath;
wherein the elongate shaft assembly comprises an outer tubular member coupled to the proximal sheath, an intermediate tubular member slidably disposed within the outer tubular member, and an inner shaft coupled to the distal sheath and slidably disposed within the intermediate tubular member;
wherein the intermediate tubular member comprises a joint disposed within the implant holding portion, the joint movably coupling a distal portion of the intermediate tubular member to a proximal portion of the intermediate tubular member.

2. The implant delivery system of claim 1, wherein the joint is configured to permit at least one degree of movement between the distal portion of the intermediate tubular member and the proximal portion of the intermediate tubular member when the distal portion of the intermediate tubular member is laterally unconstrained relative to the proximal portion of the intermediate tubular member.

3. The implant delivery system of claim 2, wherein the proximal sheath is configured to shift between a first position in which the proximal sheath laterally constrains the distal portion of the intermediate tubular member relative to the proximal portion of the intermediate tubular member, and a second position in which the proximal sheath does not laterally constrain the distal portion of the intermediate tubular member relative to the proximal portion of the intermediate tubular member.

4. The implant delivery system of claim 1, wherein the joint is configured to permit the distal portion of the intermediate tubular member to form an obtuse angle with the proximal portion of the intermediate tubular member when the distal portion of the intermediate tubular member is laterally unconstrained relative to the proximal portion of the intermediate tubular member.

5. The implant delivery system of claim 1, wherein the inner shaft comprises a lumen extending therethrough, the lumen being configured to slidably receive a guidewire therein.

6. The implant delivery system of claim 1, wherein the inner shaft extends through the joint.

7. The implant delivery system of claim 1, wherein the joint comprises a ball joint having a socket fixedly attached to the proximal portion of the intermediate tubular member and a ball fixedly attached to the distal portion of the intermediate tubular member.

8. The implant delivery system of claim 1, wherein the joint comprises a metallic tubular member extending between the proximal portion of the intermediate tubular member and the distal portion of the intermediate tubular member.

9. The implant delivery system of claim 8, wherein the metallic tubular member comprises a plurality of interconnected links cut from a monolithic tube.

10. A replacement heart valve system, comprising:
a replacement heart valve implant;
a guidewire; and
an implant delivery system for delivering the replacement heart valve implant to a native heart valve, the implant delivery system comprising an elongate shaft assembly including an implant holding portion comprising a proximal sheath and a distal sheath;
wherein in a closed configuration the implant holding portion is configured to constrain the replacement heart valve implant in a radially collapsed configuration;
wherein the elongate shaft assembly comprises an outer tubular member coupled to the proximal sheath, an intermediate tubular member slidably disposed within the outer tubular member, and an inner shaft coupled to the distal sheath and slidably disposed within the intermediate tubular member;
wherein the intermediate tubular member comprises a joint disposed within the implant holding portion, the joint movably coupling a distal portion of the intermediate tubular member to a proximal portion of the intermediate tubular member;
wherein the guidewire is slidably disposable within a lumen of the inner shaft.

11. The replacement heart valve system of claim 10, wherein the joint is configured to permit the distal portion of the intermediate tubular member to deflect laterally relative to the proximal portion of the intermediate tubular member when the distal portion of the intermediate tubular member is unconstrained by the proximal sheath.

12. The replacement heart valve system of claim 10, wherein the guidewire comprises a self-biased distal portion forming a spiral configuration when unconstrained.

13. The replacement heart valve system of claim 12, wherein when the self-biased distal portion of the guidewire is disposed within the lumen of the inner shaft and the distal portion of the intermediate tubular member is unconstrained by the proximal sheath, the distal portion of the intermediate tubular member is biased laterally by the self-biased distal portion of the guidewire.

14. The replacement heart valve system of claim 10, wherein the implant delivery system further comprises a proximal shuttle assembly disposed on the intermediate tubular member distal of the joint, the proximal shuttle assembly extending radially outward from the intermediate tubular member;
wherein engagement of the proximal shuttle assembly with the proximal sheath substantially prevents deflection of the distal portion of the intermediate tubular member relative to the proximal portion of the intermediate tubular member at the joint.

15. A method of deploying a replacement heart valve implant within a native heart valve of a patient's heart, comprising:
advancing an implant delivery system to a position adjacent the native heart valve, the implant delivery system comprising an elongate shaft assembly including an implant holding portion comprising a proximal sheath and a distal sheath configured to retain the replacement heart valve implant in a radially collapsed configuration, wherein the elongate shaft assembly comprises an outer tubular member coupled to the proximal sheath, an intermediate tubular member slidably disposed within the outer tubular member, and an inner shaft coupled to the distal sheath and slidably disposed within the intermediate tubular member, and wherein the intermediate tubular member comprises a joint disposed within the implant holding portion, the joint movably coupling a distal portion of the intermediate tubular member to a proximal portion of the intermediate tubular member;
shifting the proximal sheath from a first position to a second position, thereby releasing a proximal portion of the replacement heart valve implant;
deflecting the distal portion of the intermediate tubular member relative to the proximal portion of the intermediate tubular member at the joint; and
thereafter, shifting the distal sheath from a first position to a second position, thereby releasing a distal portion of the replacement heart valve implant.

16. The method of claim 15, wherein the distal portion of the intermediate tubular member is substantially prevented from deflecting relative to the proximal portion of the intermediate tubular member when the proximal sheath is disposed in the first position.

17. The method of claim 15, wherein the distal portion of the intermediate tubular member is substantially prevented from deflecting relative to the proximal portion of the intermediate tubular member at the joint when the proximal sheath is disposed in the first position.

18. The method of claim 15, further comprising:
positioning a guidewire comprising a self-biased distal portion forming a spiral configuration when unconstrained such that the self-biased distal portion is disposed within a left ventricle of the patient's heart; and
thereafter, advancing the implant delivery system over the guidewire to the position adjacent the native heart valve, wherein the guidewire is disposed within a lumen of the inner shaft.

19. The method of claim 18, further comprising:
after shifting the proximal sheath from the first position to the second position, withdrawing at least a portion of the self-biased distal portion of the guidewire into a lumen of the inner shaft, wherein the self-biased distal portion of the guidewire is configured to deflect the distal portion of the intermediate tubular member laterally relative to the proximal portion of the intermediate tubular member at the joint.

20. The method of claim 19, wherein rotation of the guidewire relative to the implant delivery system changes a direction of deflection of the distal portion of the intermediate tubular member relative to the proximal portion of the intermediate tubular member at the joint.
